(19) United States
(12) Patent Application Publication
(10) Pub. No.: US 2017/0009269 A1
(43) Pub. Date: Jan. 12, 2017

(54) ENHANCEMENT OF RECOMBINANT PROTEIN EXPRESSION USING A MEMBRANE-BASED CELL RETENTION SYSTEM

(71) Applicant: ADVANTECH BIOSCIENCE FARMACÊUTICA LTDA, Barueri (BR)

(72) Inventor: Sadettin Seyit OZTURK, Paoli, PA (US)

(21) Appl. No.: 15/119,202

(22) PCT Filed: Feb. 12, 2015
(86) PCT No.: PCT/BR15/00019

§ 371 Date: Aug. 16, 2016

Related U.S. Application Data
(60) Provisional application No. 61/940,493, filed on Feb. 17, 2014.

(51) Int. Cl. C12P 21/02; C12M 1/00; C12M 1/12

(52) U.S. Cl. CPC C12P 21/02; C12M 25/02; C12M 29/10

(57) ABSTRACT

The invention disclosed herein provides a novel use of an external membrane-based cell retention system in conjunction with perfusion cell culture for improved cell expression of recombinant proteins, particularly coagulation proteins such as rFVIII, B-Domain Deleted rFVIII, rFIX or rFVII/rFVIIa. The use of such a system at high cell density results in a more homogeneous cell culture due to mechanical forces induced during the operation of the retention system, such as the cell circulation induced by pumping through the fibers.

### CROSS-REFERENCE TO RELATED APPLICATIONS

This application is based on and claims priority of U.S. Ser. No. 61/940,493 filed on 17 Feb. 2014.

### FIELD

Recombinant proteins have been produced using cell culture processes that are operated either in a batch or in a continuous (perfusion) mode. A perfusion mode results in a higher volumetric yield (the amount of the product produced per volume per day) as it can sustain a higher cell density compared to a batch mode of operation.

### RELATED BACKGROUND ART

Perfusion operation has been used in the industry for the production of recombinant biological products such as monoclonal antibodies, blood factors, including coagulation proteins, enzymes, and other therapeutic proteins. The main driver for using a perfusion system is to retain the cells in the bioreactor so that the amount of product produced per volume of bioreactor is significantly enhanced. There are several types of cell retention devices on the market that have been successfully implemented in laboratory and for commercial scale production. While the increase in cell density has been demonstrated in the literature, to our knowledge, an increase in cellular productivity due to perfusion system utilizing an external cell retention membrane has not been reported.

Two references of note in this area are: 1) S. S. Ozturk and D. S. Kompala, ‘Optimization of High cell Density Perfusion Bioreactors, in Cell Culture Technology for Pharmaceutical and Cell-Based Therapies, Edited by Sadettin S. Ozturk and Wei-Shou Hu, CRC Press 2005, Pages 387-416; and 2) W. M. Woodside, B. D. Bowen, and J. M. Piret, Mammalian Cell Retention Devices for Stirred Perfusion Bioreactors, Cytotechnology, 1998, November; 28(1-3): 163-175.

The first reference above, Ozturk et al, 2005, discusses the benefits of high cell density perfusion bioreactors and the methods of handling such bioreactors in contrast to a chemostat bioreactor. A perfusion system involves the continuous inflow of nutrients coupled with the outflow of old medium while the cells are fully or partially retained within the bioreactor. One of the main advantages of perfusion bioreactors is a high protein production rate. There are many methods of cell retention, both internal to the perfusion bioreactor and external to it. Such systems, useful for heterologous cultures, are detailed in this reference and include a fixed (immobilized) bed, a ceramic matrix immobilization, hollow fiber reactors, microencapsulation and macroporous matrix fluidized systems. Cell retention systems for homogeneous cultures are often used for mammalian cell cultures in the industry because of the relative ease of scale up, monitor and control due to the uniform environment found in the suspension cultures used.

A number of cell retention devices used in mammalian cell cultures are discussed, including spin filters (internal in the bioreactor) and external filtration, such as alternating tangential flow, cell sedimentation (including vertical sedimentation and inclined sedimentation), centrifugation, ultrasonic separation and hydro cyclones.

Both flat plate and hollow fiber cartridges have been used in external filtration. Clogging of the devices can be a problem, but the external filter can be replaced when clogging reduces the flow below acceptable limits, generally between every 5 to 7 days.

The second reference given above, Woodside et al., 1998, Cytology, also discusses mammalian cell retention devices for stirred perfusion bioreactors. Woodside points out that a significant problem for a perfusion reactor design and operation is the reliability of the cell retention device, since variations in the cell culture systems can result in inconsistent post-translational modifications in large therapeutic proteins, making it necessary to demonstrate consistent process performance and product quality to obtain regulatory approval for marketing the protein.

Hollow fiber and flat plate cartridges are types of cross-flow filters. In use, the suspension from the bioreactor is pumped to an external cartridge and is concentrated as it flows across a membrane. The concentrated suspension stream is recycled to the reactor, while the cell-free permeate forms the effluent stream (See Woodside,, pgs. 164-166).

In addition to those references given above, which are considered relevant to this method, three other references have been brought to the inventor's attention. 1) US2009/0263866, a US patent application assigned to Novo Nordisk, Inc., 2) WO2011/012727, an international application assigned to Baxter Healthcare SA and Baxter International Inc., and 3) a presentation by WAVE BioTech at the FSACT 2001 in Sweden.

U.S. Ser. No. '866 is directed to Industrial Scale Serum-Free Production of Recombinant FVII in Mammalian Cells. This application outlines the various methods of cell culture used, including Batch, Fed Batch and Perfusion. A number of cell retention devices are suggested for use within the culture vessel, including an external settling head, an internal settling head, continuous centrifuge, internal or external spin filter, external filter or hollow fiber cartridge, ultrasonic cell separating device and a length of pipe inside the culture vessel (page 7, 0106-0113). The cell line used was CHO. The application focuses mainly on the uses of microcarriers as the cell retention system.

WO'725 is directed to a Method of Producing a Polypeptide or Virus of Interest in a Continuous Cell Culture. Described in this application is a ‘chemostat-like’ continuous cell culture system, which combines advantages of a perfusion open system and a chemostat open system. This hybrid system is used to culture mammalian cells. The cell retention system mentioned is a macroporous microcarrier, e.g. a cellulose-based particle.

The particular polypeptide of interest is a disintegrin-like and metallopeptidase with thrombospondin type 1 motif 13 (ADAMTS 13) protein.

The WAVE Biotech presentation discloses Perfusion Cell Culture in Disposable Bioreactors and is specifically directed to a “New Concept a floating filter”. This floating perfusion filter moves with wave motion in the interior of the Wave Bioreactor ®.

In all these references, the main focus was on the use of perfusion to increase the cell density, thus reaching higher volumetric productivity. The advantages of perfusion were discussed also for better product quality and consistency. The utility of the present invention is to increase cellular productivity and this was achieved by employing and operating an external membrane-based cell retention system in conjunction with a perfusion cell culture system.

### SUMMARY

Provided herein is a method of increasing cell expression of mammalian cells, comprising using an external membrane-based cell retention system in conjunction with a perfusion bioreactor. Preferred mammalian cells are CHO, BHK and human cells. Recombinant proteins are particularly good candidates for expression utilizing a membrane-based cell retention system. This system is useful with perfusion cell cultures to produce coagulation proteins, chosen from the group consisting of recombinant Factor IX (rFIX), recombinant Factor VIII (rFVIII), B-Domain Deleted recombinant VIII (BDD rFVIII), recombinant Factor FVII and recombinant Factor Vila (rFVII/rFVIIa). The method may be optimized by utilizing a disposable perfusion system comprising a disposable bioreactor and a disposable external membrane-based cell retention system. A preferred membrane-based cell retention system is composed of hollow fibers.

### BRIEF DESCRIPTION OF THE DRAWINGS

FIG. 1 shows the day(s) on the x-axis and the relative (normalized) titer of BDD rFVIII product on the y-axis.

FIG. 2 shows the days(s) on the x-axis and the specific productivity (normalized) on the y-axis.

### DETAILED DESCRIPTION

The membrane system was obtained from Refine Technologies (Pine Brook, N.J.). The solid squares in each figure show the results for a conventional, non-membrane-based cell retention system, BioSep, obtained from Applikon (Foster City, Calif.). The open circles in each figure show the results for the membrane-based cell retention system obtained from Refine Technologies (Pine Brook, N.J.). The bioreactors were run under the same conditions for a CHO cell line capable of producing a recombinant protein of interest, B Domain Deleted rFVIII.

This data was obtained in 2012 when ithe membrane-based retention system was used and its performance compared to a conventional, non-membrane-based cell retention system. The figures show that the membrane-based retention system clearly doubled the titer and the specific productivity of the cells. These bioreactors were run using the same cells, medium and operated using the same pH, temperature, and so forth.

The cellular productivity (the amount of product made per cell per day) is an intrinsic property of the cells and it is not expected to change with the mode of operation. Here we present a perfusion cell culture system that can actually impact the protein expression from the cells directly by controlling the cellular environment and the physiological state of the cells in the bioreactor. The present invention provides improved cellular productivity, particularly in mammalian cells, by use of an external cell retention membrane, which retains not only the cells but also cell-derived factors in the culture in conjunction with a perfusion system. This invention is applicable to many mammalian cell cultures, such as CHO, BHK, and human cell lines, particularly CHO, and to the expression of many recombinant proteins, for example antibodies and coagulation proteins such as recombinant Factor IX (rFIX), recombinant Factor VIII (rFVIII), B-Domain Deleted recombinant FVIII (BDD rFVIII), recombinant Factor VII and recombinant Factor Vila (rFVII/rFVIIa). The cell retention membrane may be a flat membrane or, preferably, a hollow fiber membrane.

The use of a membrane-based cell retention system for the cell culture of a recombinant protein was raised in 2011 during the discussions with a corporate partner. Some experts were dismissive of the idea, but testing against another (non-membrane) cell retention system, proved that it provided a significant improvement in productivity, of at least 50% and up to 100% (double).

In a preferred mode, both the bioreactor and cell retention membrane system are made of disposable materials, providing a single use, no cleaning, no steaming and no hard pipe system. The preferred cell retention membrane is external to the bioreactor and composed of hollow fibers.

Suitable disposable containers that may be used as the bioreactor may be purchased from a number of commercial sources, such as GE Healthcare, under the trade name of Wave or Xcellerex, and from ThermoFisher under the trade name Hyclone SUB and from Sartorius under the trade name Biostat STR.

Flat plate cell membranes and hollow fiber membranes for use in an external cell retention cartridge, may be sourced from GE Healthcare (Boston, Mass.), Spectrum Labs (Rancho Dominguez, Calif.), Refine Technologies (Pine Brook, N.J.), or Pall Corporation (Port Washington, N.Y.)

### EXAMPLES

Two sets of perfusion bioreactors were operated under the same conditions (pH, temperature, cell density, dissolved oxygen, volumetric flow rate, media, cell line, and so forth), one with the membrane based perfusion system (Bioreactor run 3D53), and one with a non-membrane system (Bioreactor run 3D51). Cell densities in these bioreactors were controlled at the same level and bioreactors were monitored daily for productivity, measuring the titer (product concentration). Based on cell density and bioreactor productivity, cell specific (cellular) productivities were calculated.

The membrane system was obtained from Refine Technologies (Pine Brook, N.J.). The conventional non-membrane system, BIOSEP, was obtained from Applikon (Foster City, Calif.).

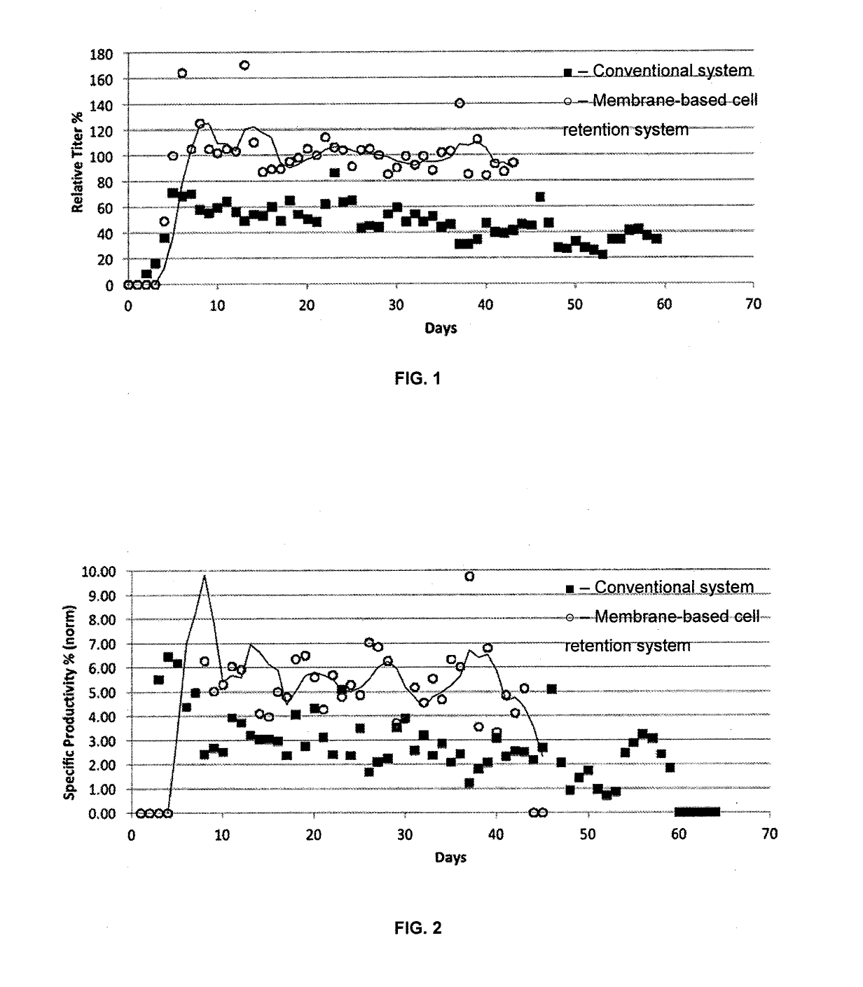

The figures show that the membrane-based retention system clearly doubled the titer and the specific productivity of the cells. The solid squares in FIGS. 1 and 2 show the results for a conventional, non-membrane-based cell retention system, BioSep, obtained from Applikon (Foster City, Calif.). The open circles in each figure show the results for the membrane-based cell retention system obtained from Refine Technologies (Pine Brook, N.J.).

Given the above disclosure, it is believed that numerous variations will occur to one skilled in the art. Therefore it is intended that the above disclosure and examples should be construed as illustrative only and that the scope of the invention should be limited only by the following claims.

What is claimed:

1. A method of increasing cell expression of mammalian cells comprising a membrane-based external cell retention system used in conjunction with a perfusion cell culture.

2. The method of claim 1, wherein the method of increasing cell expression of mammalian cells is used to produce recombinant proteins.

3. The method of claim 2, wherein the recombinant proteins expressed are coagulation proteins.

4. The method of claim 3, wherein the expressed coagulation proteins are chosen from the group consisting of recombinant Factor IX, recombinant FVIII, B-Domain-Deleted recombinant Factor FVIII, recombinant VII and recombinant Factor VIIa.

5. The method of claim 1, wherein the mammalian cells are chosen from CHO, BHK and mammalian cells.

6. The method of claim 5, wherein the membrane based external cell retention system used in conjunction with perfusion cell culture is capable of expressing B-Domain Deleted recombinant Factor VIII and provides an increase in production of B-Domain Deleted recombinant FVIII protein of at least 50% over that obtained with a cell retention system not utilizing a membrane filtration system.

7. The method of claim 1, wherein the perfusion cell culture is performed in a disposable bioreactor.

8. The method of claim 1, wherein the membrane-based cell retention system is disposable.

9. The method of claim 1, wherein the cell culture system is disposable because both the bioreactor that contains the perfusion system and the external membrane-based cell retention system are disposable.
